(19) United States
(12) Patent Application Publication
(10) Pub. No.: US 2023/0393129 A1
(43) Pub. Date: Dec. 7, 2023

(54) IMMUNOCHROMATOGRAPHIC KIT FOR FLUORESCENT IMMUNOCHROMATOGRAPHY AND HOUSING CASE COMPRISING IMMUNOCHROMATOGRAPHIC KIT

(71) Applicant: National University Corporation Saitama University, Saitama-shi, Saitama (JP)

(72) Inventors: Ken HATANO, Saitama-shi, Saitama (JP); Koji MATSUOKA, Saitama-shi, Saitama (JP); Takahiko MATSUSHITA, Saitama-shi, Saitama (JP)

(21) Appl. No.: 18/037,322

(22) PCT Filed: Nov. 25, 2021
(86) PCT No.: PCT/JP2021/043264

§ 371 Date: May 17, 2023

(30) Foreign Application Priority Data
Nov. 26, 2020 (JP) 2020-195626

(51) Int. Cl. G01N 33/543; G01N 21/64

(52) U.S. Cl. CPC G01N 33/54387; G01N 21/6428; G01N 2021/6439

(57) ABSTRACT

Provided with a sheet and a backing sheet for reducing a background luminescence occurs in a fluorescent immunochromatography method. In an immunochromatographic kit having at least a membrane and a backing sheet, wherein a luminescence suppression sheet is provided between the membrane and the backing sheet, and a quantum yield measured by an absolute PL quantum yield spectrometer C11347 manufactured by Hamamatsu Photonics K.K. is 0.40% or less when the luminescence suppression sheet is irradiated with an excitation light having a wavelength of 365 nm.

### TECHNICAL FIELD

The present invention relates to an immunochromatographic kit and a housing case comprising the immunochromatographic kit for reducing a background luminescence (light emission) generated when performing a fluorescent immunochromatography method.

### BACKGROUND ART

The fluorescent immunochromatography method is frequently used for a primary inspection as a method of measuring an infection quickly and easily. Conventionally, the method of using gold colloid was mainly used. However, the problem was that the sensibility was low. As an effective means of solving the above described problem, it can be considered that the gold colloid used as a labeled substance is replaced with a luminescent substance. However, in a fluorescent immunochromatography method using a fluorescent substance, an ultraviolet ray irradiation is necessary for exciting the fluorescent substance, different from the gold colloid which can be judged only by visual observation without using special tools and methods. However, the ultraviolet ray irradiation has not only the property of making the fluorescent substance emit light but also the property of making the components of the immunochromatographic kit such as a backing sheet (base portion) and a membrane (development portion) emit light. Accordingly, when detecting weak fluorescence emitted from a small amount of antigen, for example, an SN ratio is deteriorated and an antibody line is difficult to appear clearly.

For solving the above described problem, several methods shown in Patent Documents 1 to 3 are proposed. For example, the method of using colored cellulose fine particles having uniform particle size (Patent Document 1), the method of using an immunochromatography device where a space is formed between a conjugate pad and a membrane (Patent Document 2), and the method of using a pigment having the color which has a relation of complementary color with the color of a labeled carrier (Patent Document 3) are proposed.

It is considered that the all of the above described methods have the effect of reducing the background luminescence. However, all of the above described methods have the problem that the operations are complicated, for example. Thus, the necessity of the method of reducing the background luminescence more easily is still high.

### PRIOR ART DOCUMENTS

### Patent Documents

Patent Document 1: Japanese Unexamined Patent Application Publication No. 2020-125909
Patent Document 2: Japanese Unexamined Patent Application Publication No. 2013-205336
Patent Document 3: Japanese Unexamined Patent Application Publication No. 2016-206117

### DISCLOSURE OF THE INVENTION

### Problems to be Solved by the Invention

Considering the above described situation, the first to seventh inventions of the present application provides a method for reducing a background luminescence generated when performing a fluorescent immunochromatography method, especially for performing a fluorescent immunochromatography method including the process of irradiating luminescent molecules with an excitation light to detect the luminescence, and a fluorescent immunochromatography device used for the fluorescent immunochromatography method.

### Means for Solving the Problem

An immunochromatography device used for a general fluorescent immunochromatography method has a backing sheet (51), a membrane (52), an absorption pad (53), a conjugate pad (54) and a sample pad (55) in a housing case (50) as shown in FIG. 7. In the housing case (50), as shown in FIG. 7, the sample pad (55), the conjugate pad (54) and the absorption pad (53), the membrane (reaction film) (52) to which capture antibody (antibody specifically binding to an antigen or a primary antibody to be detected) is fixed, and the backing sheet (51) are laminated in this order from the above.

When a sample solution containing an antigen and the like is added to the sample pad (55), the sample solution is transferred to the conjugate pad (54) which contains a labeled primary antibody. Thus, the antigen is bound to the labeled primary antibody. The composite of the labeled primary antibody and the antigen is transferred on the membrane (52) by capillary action and captured by a secondary antibody (an antibody bound to the antigen) which is fixed to a test line. On the other hand, the labeled primary antibody not bound to the antigen passes through the test line and is captured by a control secondary antibody (an antibody which recognizes the primary antibody without being bound to the antigen) which is fixed to a control line. When the antigen to be detected is contained in a specimen (test sample), the luminescence derived from the label is detected from both the test line and the control line. When the antigen is not contained in the specimen, the luminescence derived from the label is detected only from the control line.

Here, the inventors of the present application found that the background luminescence could be suppressed by using a sheet, a backing sheet or a housing case having a certain property when the membrane is irradiated with light. The above described “certain property” is related to a quantum yield or a color of the sheet, the backing sheet or the housing case. It is revealed that the background luminescence of the membrane can be efficiently suppressed when the above described quantum yield is less than a predetermined value or the color is black.

The first to seventh inventions of the present application are as follows.

### First Invention

An immunochromatographic kit having at least a membrane and a backing sheet, wherein a luminescence suppression sheet is provided between the membrane and the backing sheet, and a quantum yield measured by an absolute PL quantum yield spectrometer C11347 manufactured by Hamamatsu Photonics K.K. is 0.40% or less when the luminescence suppression sheet is irradiated with an excitation light having a wavelength of 365 nm.

### Second Invention

An immunochromatographic kit having at least a membrane and a backing sheet, wherein a luminescence suppression sheet is provided between the membrane and the backing sheet, and a quantum yield measured by an absolute PL quantum yield spectrometer C11347 manufactured by Hamamatsu Photonics K.K. is 0.40% or less when the luminescence suppression sheet is irradiated with an excitation light having a wavelength of 254 nm.

### Third Invention

An immunochromatographic kit having a membrane and a backing sheet, wherein a quantum yield measured by an absolute PL quantum yield spectrometer C11347 manufactured by Hamamatsu Photonics K.K. is 0.40% or less when the backing sheet is irradiated with an excitation light having a wavelength of 365 nm.

### Fourth Invention

An immunochromatographic kit having a membrane and a backing sheet, wherein a quantum yield measured by an absolute PL quantum yield spectrometer C11347 manufactured by Hamamatsu Photonics K.K. is 0.40% or less when the backing sheet is irradiated with an excitation light having a wavelength of 254 nm.

### Fifth Invention

An immunochromatography device, wherein an immunochromatographic kit having a membrane and a backing sheet is placed on a surface of a bottom plate of a housing case, and a quantum yield measured by an absolute PL quantum yield spectrometer C11347 manufactured by Hamamatsu Photonics K.K. is 0.40% or less when the immunochromatography device is irradiated with an excitation light having a wavelength of 365 nm.

### Sixth Invention

An immunochromatography device, wherein the immunochromatographic kit of the first invention or the second invention is arranged on a black housing case.

### Seventh Invention

A method of detecting an antibody line shown on a membrane after a specimen is dropped on the membrane, wherein the membrane is irradiated with a light in a state that the luminescence suppression sheet of the first invention or the second invention is arranged between the membrane and the backing sheet, or in a state that the backing sheet of the third invention or the fourth invention is arranged below the membrane, or in a state that the housing case of the fifth invention or the sixth invention is arranged below the membrane and the backing sheet.

### Effects of the Invention

In the fluorescent immunochromatography method using a fluorescent molecule or the like as a labeled substance, the background luminescence of the membrane can be suppressed when the luminescence suppression sheet is provided between the membrane and the backing sheet and the quantum yield of the luminescence suppression sheet is 0.40% or less when the luminescence suppression sheet is irradiated with the excitation light having a wavelength of 365 nm and the quantum yield of the luminescence suppression sheet is 0.40% or less when the luminescence suppression sheet is irradiated with an excitation light having a wavelength of 254 nm as shown in the first and second inventions. Accordingly, the visibility of the luminescence emitted from the test line is improved and the measurement can be performed with high sensitivity.

In the third and fourth inventions, the background luminescence of the membrane can be suppressed when the quantum yield is 0.40% or less when the backing sheet is irradiated with the excitation light having a wavelength of 365 nm and the quantum yield is 0.40% or less when the backing sheet is irradiated with the excitation light having a wavelength of 254 nm as described above. Accordingly, the visibility of the luminescence emitted from the test line is improved and the measurement can be performed with high sensitivity.

In the fifth and sixth inventions, the background luminescence of the membrane can be suppressed when the immunochromatographic kit of the first invention and the second invention is arranged on a black housing case as described above. Accordingly, the visibility of the luminescence emitted from the test line is improved and the measurement can be performed with high sensitivity.

### BRIEF DESCRIPTION OF THE DRAWINGS

FIG. 1 is a conceptual diagram showing an immunochromatography device of Embodiment 1.

FIG. 2 shows images when the immunochromatography device of Embodiment 1 is irradiated with an excitation light.

FIG. 3 is a conceptual diagram showing an immunochromatography device of Embodiment 2.

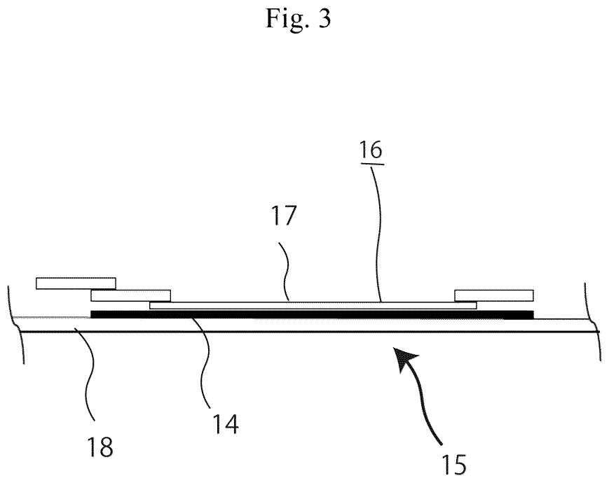

FIG. 4 shows images when the immunochromatography device of Embodiment 2 is irradiated with an excitation light.

FIG. 5 is a conceptual diagram showing an immunochromatography device of Embodiment 3.

FIG. 6 shows images when the immunochromatography device of Embodiment 3 is irradiated with an excitation light.

FIG. 7 is a conceptual diagram showing an immunochromatography device of the conventional example.

### MODES FOR CARRYING OUT THE INVENTION

### Embodiment 1

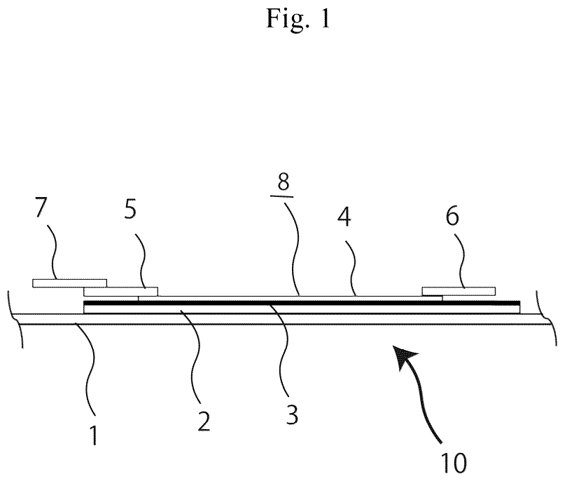

Embodiment 1 which is based on the first, second and seventh inventions of the present application will be explained below. In the present embodiment, as shown in FIG. 1, a backing sheet 2, 14, 21, a luminescence suppression sheet 3 and a membrane 4, 17, 20 are laminated on a surface of a bottom plate 1, 18, 23 of a housing case in this order. In addition, a conjugate pad 5 is arranged on a surface 8, 16, 22 of one end of the membrane 4, 17, 20 and an absorption pad 6 is arranged on the surface 8, 16, 22 of the other end of the membrane 4, 17, 20. Furthermore, a sample pad 7 is arranged on a surface of one end of the conjugate pad 5. The backing sheet 2, 14, 21, the luminescence suppression sheet 3, the membrane 4, 17, 20, the conjugate pad 5, the absorption pad 6 and the sample pad 7 constitute an immunochromatographic kit 10, 15, 24 of the present embodiment. In the present embodiment, as shown in FIG. 1, one luminescence suppression sheet is laminated and arranged between the membrane and the backing sheet. However, in the different embodiments, it is possible to laminate a plurality of luminescence suppression sheets.

It is preferable that the other components of the immunochromatography device are not interposed between the membrane and the luminescence suppression sheet except for the component integrated with the membrane. In addition, the luminescence suppression sheet is arranged between the membrane and the backing sheet before a specimen is collected in the present embodiment. However, it is also possible to arrange the luminescence suppression sheet between the membrane and the backing sheet only when detecting an antibody line.

In the present embodiment and other embodiments, “membrane” means a reaction film to which the antibody is fixed, “backing sheet” means a support member for supporting the membrane. The luminescence suppression sheet of the present embodiment can be used for reducing the background luminescence derived from the immunochromatography device and the like and improving the detection sensitivity when the antigen (hereafter, also referred to as “detecting antigen”) in the sample is detected by the fluorescent immunochromatography.

Namely, it is required to reduce the background luminescence for improving the visibility of the luminescence derived from the label when the labeled antibody bound to the detecting antigen exists in the test line. Therefore, the membrane was irradiated with an excitation light (light for exciting the labeled molecules bound to the antibody) in a state that the luminescence suppression sheet of the present embodiment was arranged below the membrane as shown in FIG. 1. As a result, it was revealed that the background luminescence derived from the immunochromatography device and the like was suppressed and the visibility of the luminescence derived from the label bound to the antibody was improved.

The shape of the luminescence suppression sheet of the present embodiment is not particularly limited since the shape depends on the configuration of the immunochromatography device to which the luminescence suppression sheet is attached. In addition, the thickness of the luminescence suppression sheet is not particularly limited. For example, the luminescence suppression sheet can be used when the thickness is within the range of approximately 0.005 mm to 1 mm. In addition, the material of the luminescence suppression sheet is not particularly limited. For example, polyvinyl chloride, cellulose, polyethylene and a mixture of them are exemplified as the material. Any materials can be used as long as the quantum yield of the luminescence suppression sheet is within the later described range. In addition, the luminescence suppression sheet can contain paint components. As a specific example of the luminescence suppression sheet, commercially available vinyl tapes and masking tapes are exemplified, for example. In addition, an adhesive agent and the like can be applied on the luminescence suppression sheet for fixing the luminescence suppression sheet to the other components (e.g., backing sheet) of the immunochromatography device.

(Measurement of Quantum Yield of Luminescence Suppression Sheet)

First, the quantum yield of the luminescence suppression sheet of the present embodiment was measured. As the luminescence suppression sheet to be measured, Sample 1 (black) and Sample 2 (white) using a vinyl tape (Horyku) and Sample 3 (green), Sample 4 (red), Sample 5 (blue), Sample 6 (cream color) and Sample 7 (pink) using a masking tape (Copeflap) were adopted. Each of the above described samples was cut into a square shape having sides of approximately 1 cm and placed on a petri dish for measurement of a quantum yield measuring device (Quantaurus-QY Absolute PL quantum yield spectrometer C11347, Hamamatsu Photonics K.K., Japan) so that the adhesive portion of the tape faced downward. Then, the petri dish for measurement was set to a sample holder and the quantum yield of each sample was measured. As the measurement condition, solid mensuration was performed at room temperature and a wavelength of the excitation light was 365 nm or 254 nm. The relation between each of the samples and the quantum yield in the above described measurement result is shown in Table 1. In the measurement result, the tendency was similar between 365 nm and 254 nm. As shown in Table 1, the quantum yield of Samples 1 and 3 to 5 was 0.4% or less, which was relatively low value, while the quantum yield of Samples 2, 6 and 7 was 1.30% or more, which was high value.

TABLE 1

| | quantum yield | |
|---|---|---|
| | 365 nm | 254 nm |
| Sample 1 | 0% | 0% |
| Sample 2 | 10% | 6.40% |
| Sample 3 | 0.20% | 0.40% |
| Sample 4 | 0.30% | 0.30% |
| Sample 5 | 0.40% | 0.20% |
| Sample 6 | 4.30% | 7.90% |
| Sample 7 | 1.30% | 5.90% |

(Measurement of Quantum Yield of Immunochromatographic Kit)

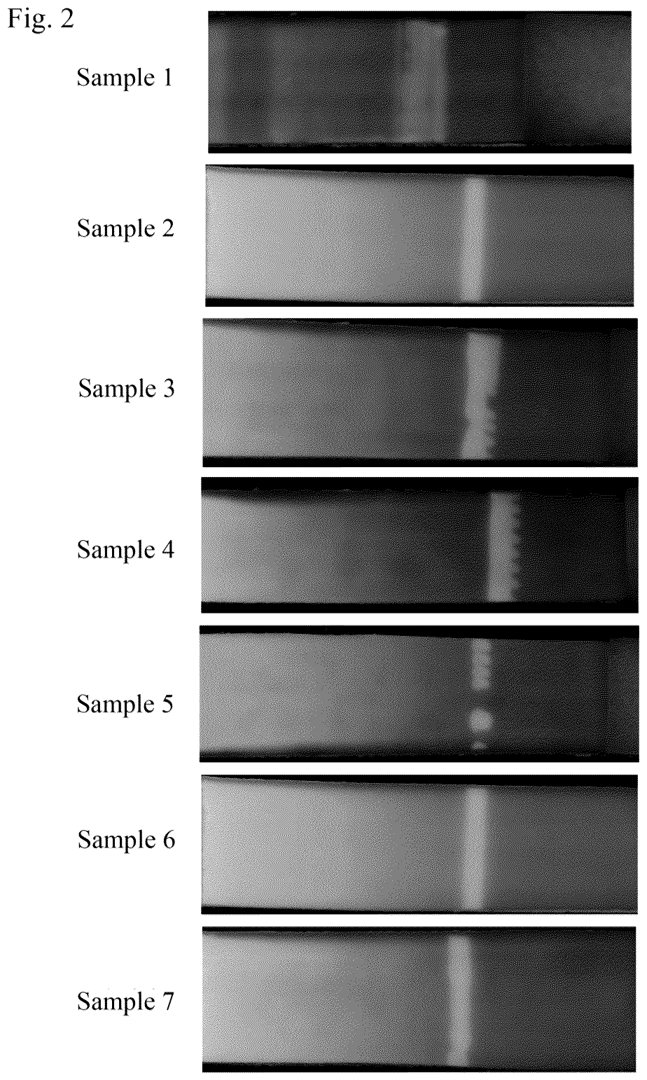

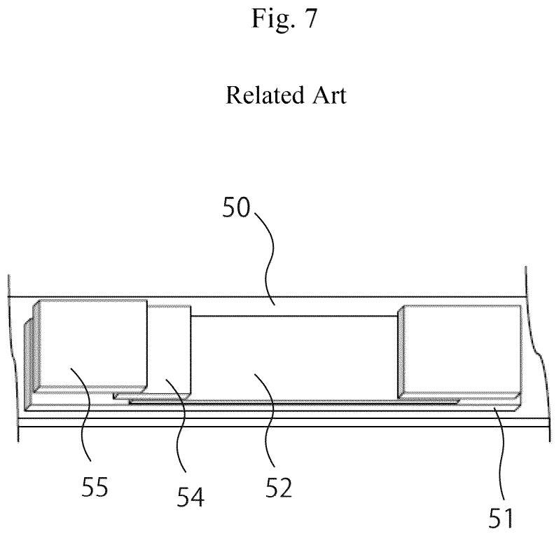

Then, a measurement experiment of the quantum yield was performed when the membrane not including an antibody line was irradiated with the excitation light (wavelength 365 nm). Before performing the measurement, the immunochromatographic kit comprising the membrane to which the antibody is not bound, various tapes and the backing sheet was prepared. Namely, the backing sheet (Lohmann (G&L)) was arranged and each of the samples (Samples 1 to 7) was adhered to the center portion of the backing sheet. The membrane was adhered to each of the samples so that the membrane was exactly overlapped with each of the samples. Thus, the immunochromatographic kit comprising the membrane, each of the samples and the backing sheet was prepared (shown in FIG. 2). In addition, as shown in FIG. 7, the conventional immunochromatographic kit where the membrane was directly arranged on the surface of the backing sheet without arranging the sample was also prepared similarly.

The measurement result is shown in Table 2 below.

TABLE 2

| | quantum yield |
|---|---|
| No sample | 9.60% |
| Sample 1 | 5.00% |
| Sample 2 | 7.30% |
| Sample 3 | 5.30% |
| Sample 4 | 5.10% |
| Sample 5 | 4.80% |
| Sample 6 | 7.20% |
| Sample 7 | 6.00% |

From Table 2, it was confirmed that the quantum yield of the immunochromatographic kit using Samples 1 and 3-5, which were the samples where the quantum yield was low in Table 1, was reduced to approximately half compared to the immunochromatographic kit without arranging the sample.

Furthermore, the visibility was confirmed about how much the background luminescence of the membrane bound to the antibody is reduced using Samples 1 to 7.

(Manufacture of Membrane to which Antibody is Bound)

An anti-mouse IgG rabbit antibody (Immuno Probe Co., Ltd.) was used as the primary antibody. PBS (1.5 mg/mL) containing the primary antibody was linearly added to the membrane (support member: polyethylene terephthalate (PET) film) using a stainless steel pipe, the PBS was dried at 60° C. for 30 minutes using a drying oven, and the membrane was soaked in a blocking buffer (1.0% casein, 0.1 boric acid buffer pH8.5) and left stably for 30 minutes. The membrane to which blocking was performed was soaked in a washing buffer (10.0% sucrose, 0.1% sodium cholate, 0.1 M tris (hydroxymethyl) aminoethane buffer pH 7.5) and shaken for 30 minutes, excess water was wiped off and the membrane was dried under the humidity of 20% or less during a full night.

(Manufacture of Immunochromatographic Kit Comprising Membrane, Each Sample and Backing Sheet)

The backing sheet (Lohmann (G&L)) was prepared so that the adhesive portion faced upward and each of Samples 1 to 7 was adhered to the center portion of the backing sheet so that the adhesive portion faced upward. Then, the membrane to which the antibody was fixed as described above was adhered on each of the adhered samples so that the membrane was exactly overlapped with the tape. Thus, the immunochromatographic kit comprising the membrane, each of the samples and the backing sheet was prepared. Similarly, the conventional immunochromatographic kit to which the above described sample is not adhered was also prepared.

(Confirmation of Visibility of Antibody Line on Membrane)

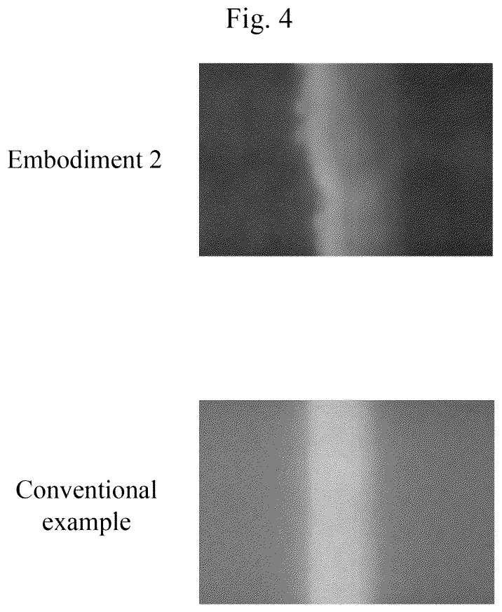

Each immunochromatographic kit prepared by the above described method was housed in the housing case, the immunochromatographic kit was reacted with PBS (100 μg/mL) containing a mouse IgG (anti-influenza A virus antibody, used as a secondary antibody) which is labeled by fluorescence. The fluorescent label used here was polystyrene beads containing fluorescent pigment having the maximum fluorescence wavelength of 510 nm. In addition, silole derivative was used for the fluorescent pigment (shown in Yamaguchi, et al., Chem. Eur. J., 6(9): 1683-1692 2000). After the reaction was finished, the immunochromatographic kit was set to a fluorescence reader (Musashi Optical System Co., Ltd.) and the membrane was irradiated with the excitation light (wavelength 365 nm) to observe the antibody line. The observed images are shown in FIG. 4.

As shown in FIG. 2, it was visually confirmed that the background luminescence of the membrane was reduced when each of the samples was arranged between the membrane and the backing sheet compared to the conventional example where the sample was not arranged. In particular, it was confirmed that the effect of reducing the background luminescence was more remarkable when the luminescence suppression sheet having small quantum yield like Samples 1 and 3 to 5 shown in Table 1 was used.

### Embodiment 2

It is known that the background luminescence emitted from the backing sheet constituting the conventional immunochromatography device becomes a factor for reducing the detection sensitivity. From the above described fact, it is considered that the background luminescence derived from the backing sheet can be suppressed if the quantum yield of the backing sheet itself is almost same as the quantum yield of the luminescence suppression sheet of Embodiment 1. Accordingly, it can be said that the effect of reducing the background luminescence can be obtained if the backing sheet having the quantum yield of 0.4% or less when the backing sheet is irradiated with the excitation light of 365 nm is used or the backing sheet having the quantum yield of 0.4% or less when the backing sheet is irradiated with the excitation light of 254 nm is used similar to Embodiment 1.

Then, Embodiment 2 based on the third, fourth and seventh inventions will be explained below. Note that “backing sheet” of the present embodiment is the component constituting the bottom surface of the immunochromatography device as described above. More specifically, the backing sheet is the component used for the support member of the immunochromatographic kit. For example, the backing sheet can have a sheet shaped structure formed of polyethylene, terephthalate, polystyrene, polypropylene, polyester, polyvinyl chloride or a mixture of them. An adhesive agent or the like can be applied on an upper surface of the backing sheet.

Furthermore, the backing sheet can contain paint components. The backing sheets having various shapes (e.g., length, width and thickness) can be adopted depending on the type of the immunochromatography device to be used. For example, the thickness of the backing sheet can be approximately 0.1 mm to 1.0 mm although the thickness is not particularly limited.

The visibility was confirmed in the following procedures about the effect of reducing the background luminescence of the membrane when the backing sheet having low quantum yield was used. In the present embodiment, Sample 1 was used as the backing sheet since Sample 1 showed the good result in the above described Embodiment 1. The quantum yield of Sample 1 is shown in Table 1.

(Manufacture of Membrane to which Antibody is Bound)

An anti-mouse IgG rabbit antibody (Immuno Probe Co., Ltd.) was used as the primary antibody. PBS (1.5 mg/mL) containing the primary antibody was linearly added to the membrane (support member: polyethylene terephthalate (PET) film) using a stainless steel pipe, the PBS was dried at 60° C. for 30 minutes using a drying oven, and the membrane was soaked in a blocking buffer (1.0% casein, 0.1 boric acid buffer pH8.5) and left stably for 30 minutes. The membrane to which blocking was performed was soaked in a washing buffer (10.0% sucrose, 0.1% sodium cholate, 0.1 M tris (hydroxymethyl) aminoethane buffer pH 7.5) and shaken for 30 minutes, excess water was wiped off and the membrane was dried under the humidity of 20% or less during a full night.

(Manufacture of Immunochromatographic Kit Constituting Membrane and Backing Sheet)

Sample 1, which was the backing sheet of the present embodiment, was arranged so that the adhesive portion faced upward and the membrane was adhered to Sample 1 so that the membrane was exactly overlapped with Sample 1. Thus, the immunochromatographic kit constituting the membrane and Sample 1 was prepared. Similarly, the immunochromatographic kit using the conventional backing sheet was prepared.

(Measurement of Quantum Yield of Immunochromatographic Kit)

First, the quantum yield when the membrane of the immunochromatographic kit without containing an antibody line was irradiated with the excitation light (wavelength 365 nm) was measured similar to the above described Embodiment 1. The result is shown in Table 3. In table 3, “Embodiment 2” means the immunochromatographic kit using the backing sheet of Sample 1, while “Conventional example” means the immunochromatographic kit using the conventional backing sheet (Lohmann (G&L)).

TABLE 3

| | quantum yield |
|---|---|
| Sample 2 | 0.60% |
| Conventional example | 9.60% |

From table 3, it was confirmed that the quantum yield of Embodiment 2 was significantly low when compared with Conventional example. The above described result shows the same tendency as the experiment result of the luminescence suppression sheet of the above described Embodiment 1. It is suggested that the background luminescence of the membrane can be suppressed more efficiently by using the backing sheet having lower quantum yield.

(Confirmation of Visibility of Antibody Line on Membrane)

The immunochromatographic kits of Embodiment 2 and Conventional example were reacted with PBS (100 μg/mL) containing a mouse IgG (anti-influenza A virus antibody, used as a secondary antibody) which is labeled by fluorescence in a state of being housed in the housing case. The fluorescent label used here was polystyrene beads containing fluorescent pigment having the maximum fluorescence wavelength of 510 nm. In addition, silole derivative was used for the fluorescent pigment (shown in Yamaguchi, et al., Chem. Eur. J., 6(9): 1683-1692 2000). After the reaction was finished, each of the immunochromatographic kits was set to a fluorescence reader (Musashi Optical System Co., Ltd.) and the membrane was irradiated with the excitation light (wavelength 365 nm) to observe the antibody line. The observed images are shown in FIG. 4. From FIG. 4, it could be confirmed that the effect of reducing the background luminescence of the membrane was remarkable in Embodiment 2 compared to Conventional example.

### Embodiment 3

It is also considered that the effect of reducing the background luminescence of the membrane can be further obtained depending on the color of the housing case housing the immunochromatographic kit of the above described Embodiments 1 and 2 inside. Therefore, the visibility was confirmed in the following procedures about the immunochromatography device where the immunochromatographic kit constituting Sample 1 used in the above described Embodiment 1, the membrane and the backing sheet was housed in a black housing case and a white housing case.

(Manufacture of Membrane to which Antibody is Bound)

An anti-mouse IgG rabbit antibody (Immuno Probe Co., Ltd.) was used as the primary antibody. PBS (1.5 mg/mL) containing the primary antibody was linearly added to the membrane (support member: polyethylene terephthalate (PET) film) using a stainless steel pipe, the PBS was dried at 60° C. for 30 minutes using a drying oven, and the membrane was soaked in a blocking buffer (1.0% casein, 0.1 boric acid buffer pH8.5) and left stably for 30 minutes. The membrane to which blocking was performed was soaked in a washing buffer (10.0% sucrose, 0.1% sodium cholate, 0.1 M tris (hydroxymethyl) aminoethane buffer pH 7.5) and shaken for 30 minutes, excess water was wiped off and the membrane was dried under the humidity of 20% or less during a full night.

(Manufacture of Immunochromatography Device)

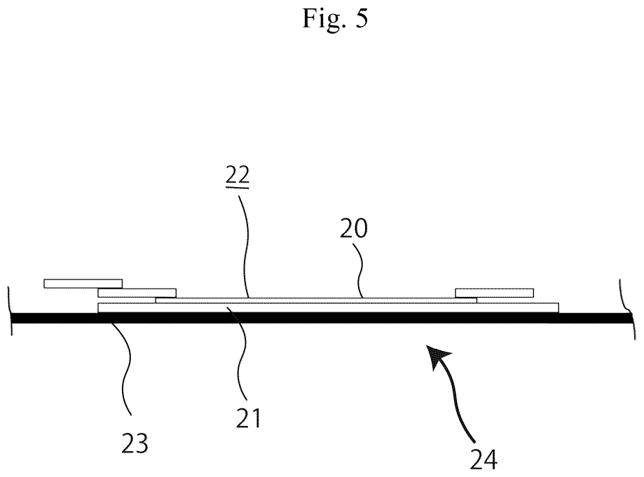

The conventional backing sheet (Lohmann (G&L)) was arranged so that the adhesive portion faced upward and each of Samples 1 to 7 was adhered to the center portion of the backing sheet so that the adhesive portion faced upward. The membrane was adhered to each of the samples so that the membrane was exactly overlapped with each of the samples. Thus, the immunochromatographic kit comprising the membrane, the luminescence suppression sheet of Sample 1 and the backing sheet was prepared (shown in the figure). Then, the immunochromatographic kit was housed in the black housing case and the white housing case shown in FIG. 5. Thus, the immunochromatography device was prepared.

(Confirmation of Visibility of Antibody Line on Membrane)

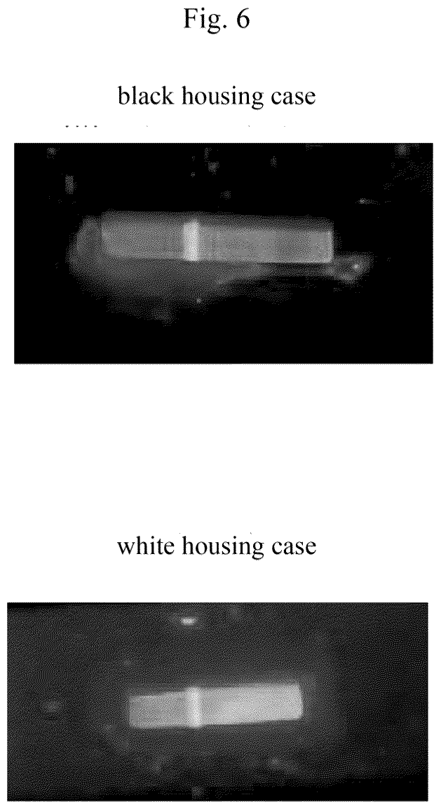

The immunochromatographic kit prepared by the above described method was reacted with PBS (100 μg/mL) containing a mouse IgG (anti-influenza A virus antibody, used as a secondary antibody) which is labeled by fluorescence. The fluorescent label used here was polystyrene beads containing fluorescent pigment having the maximum fluorescence wavelength of 510 nm. In addition, silole derivative was used for the fluorescent pigment (shown in Yamaguchi, et al., Chem. Eur. J., 6(9): 1683-1692 2000). After the reaction was finished, each of the immunochromatographic kits was set to a fluorescence reader (Musashi Optical System Co., Ltd.) and the membrane was irradiated with the excitation light (wavelength 365 nm) to observe the antibody line. The observed images are shown in FIG. 6. From FIG. 6, it could be confirmed that the effect of reducing the background luminescence of the membrane was further remarkable when the black housing case was used compared to when the white housing case was used.

### INDUSTRIAL APPLICABILITY

The first to seventh inventions of the present application have the effect of improving the detection sensitivity using the immunochromatography. Accordingly, the present application is effective when performing various diagnoses using the fluorescent immunochromatography method.

1. An immunochromatographic kit having at least a membrane and a backing sheet, wherein
a luminescence suppression sheet is provided between the membrane and the backing sheet, and
a quantum yield is 0.40% or less when the luminescence suppression sheet is irradiated with an excitation light having a wavelength of 365 nm.

2. An immunochromatographic kit having at least a membrane and a backing sheet, wherein
a luminescence suppression sheet is provided between the membrane and the backing sheet, and
a quantum yield is 0.40% or less when the luminescence suppression sheet is irradiated with an excitation light having a wavelength of 254 nm.

3-4. (canceled)

5. An immunochromatography device, wherein
an immunochromatographic kit having a membrane and a backing sheet is placed on a surface of a bottom plate of a housing case, and
a quantum yield is 0.40% or less when the immunochromatography device is irradiated with an excitation light having a wavelength of 365 nm.

6. An immunochromatography device, wherein the immunochromatographic kit of claim 1 is arranged on a black housing case.

7. A method of detecting an antibody line shown on a membrane after a specimen is dropped on the membrane, wherein
the membrane is irradiated with a light in a state that the luminescence suppression sheet of claim 1 is arranged between the membrane and the backing sheet.
